(19) United States
(12) Patent Application Publication
(10) Pub. No.: US 2022/0023473 A1
(43) Pub. Date: Jan. 27, 2022

(54) DISTRIBUTED STERILIZER SYSTEM

(71) Applicant: MEDKLINN TECHNOLOGY PTE. LTD., Singapore (SG)

(72) Inventor: Kok Wah LU, Kuala Lumpur (MY)

(21) Appl. No.: 17/276,859

(22) PCT Filed: Oct. 11, 2018
(86) PCT No.: PCT/SG2018/000005

§ 371 Date: Mar. 17, 2021

(51) Int. Cl. A61L 2/18; A61L 9/015; C01B 13/10

(52) U.S. Cl. CPC A61L 2/183; A61L 9/015; C01B 13/10; A61L 2209/212; A61L 2209/11; A61L 2202/11; A61L 2202/14; A61L 2209/13; A61L 2202/15

(57) ABSTRACT

A distributed sterilizer system for distributing ozone water to a plurality of rooms comprising an ozone generation system for generating ozone from an air supply, a plurality of injection devices for mixing the ozone generated from the ozone generation system with a supply of water to form ozone water and a delivery piping system for distributing the ozone water from the plurality of injection devices into each of the plurality of rooms. The ozone generation system further comprises a centralized oxygen concentrator for providing a supply of oxygen and a plurality of ozone generators for generating ozone from the oxygen supplied by the centralized oxygen concentrator. The distributed sterilizer system also provides a mechanism for distributing ozone directly to a plurality of other rooms.

### FIELD OF INVENTION

The invention relates generally to a sterilizer system for performing effective sterilization using ozone water and ozone. More specifically, the invention concerns a sterilizer system for distributing a controlled level of ozone water and ozone to large indoor areas and/or in multiple locations at one time.

### BACKGROUND OF THE INVENTION

At present day, depending on the type of application, it is common to use hot water, normal water, detergents or enzyme to remove smells, bacteria or other pollutants. For applications in toilet, normal chlorine water and detergents are commonly used to clean surfaces, while artificial fragrance is used to mask the smell in the air. In the food industry especially kitchen or central kitchen, a combination of hot water, detergents and enzyme is frequently used for surface cleaning. For applications of surrounding air in the kitchen, ionizers or UV lights are known to be used but such products are found to be not effective as it should be.

Ozone is a strong oxidant and potent disinfecting agent which is commonly used for sterilization because of its strong oxidizing properties. Ozone is approved by the FDA and USDA governing authorities for used in food industry. Therefore, it is with great interest to look into maximizing the potential usage of ozone in the food industry while expanding its applications in other areas such as toilets, refuse chambers and bin centers. It is common knowledge that ozonated water is used for a wide variety of cleaning applications. However, the existing delivery method of these ozonated water is limited to certain scales and types of applications with an average efficiency. There is a great interest to use ozone water in a larger scale such as a central ozone water system for cleaning in multiple locations at one time. For example, in a central kitchen it has many sections such as preparation room, butchery room, vegetable processing room and packaging room. Similarly, in public toilets where there is female toilet, male toilet, handicap toilet and baby changing room, the end users would want to use ozone water in these mentioned areas using a central system. It is common knowledge that large kitchens and public toilets have bad odor problems and hygiene concerns. While ozone water is an effective method to sterilize surfaces to eliminate bacteria and deodorize the smells coming from the kitchen surface or toilet smell from urine on the floor, it is not effective to sterilize airborne pollutants. The usage of ozone is crucial in dealing with this problem. Therefore, there is a great need for an integrated distribution system to generate and distribute ozone water and ozone effectively.

US patent publication no. 2013/0224077 A1 discloses a distributed ozone disinfection system having a central ozone generation system, and ozone and water mixing systems. Each of the ozone and water mixing systems is positionable in a water supply piping at a water supply inlet for a sink faucets or water outlets. The distributed ozone disinfection system has vacuum switches, separate from vacuum switches positionable downstream which are in turn separate from the ozone and water mixing systems, and a plurality of oxidation reduction potential (ORPs) meters. The ORP meters are positionable downstream and separate from the ozone and water mixing systems. Optionally, the ozone and water mixing system includes a vacuum switch coupled with a gas injection venturi device.

U.S. Pat. No. 6,343,779 B1 discloses a water distribution piping for gas-dissolved cleaning water which distributes cleaning water, made by dissolving gas in pure water, in the presence of gas, the piping having a main pipe and branch pipes, including an in-line mixer immediately upstream of each point at which a branch pipe extends from the main pipe. Ozone dissolves in water to form ozone-dissolved cleaning water which flows through a main pipe. The water distribution piping has an in-line mixer immediately upstream of a branching point where a branch pipe branches off from the main pipe.

The sterilizer systems as disclosed in both US patent publication no. 2013/0224077 A1 and U.S. Pat. No. 6,343,779 B1 solely focus on the generation of ozone water to be distributed to multiple locations. Both systems are unable to accommodate a mechanism for distributing ozone as well in addition to distributing ozone water to multiple locations. In addition, there is a need for more flexibility in controlling the formation and distribution of ozone and ozone water to multiple locations based on a time-based configuration and a demand-based configuration. This flexibility of control is not evident in the systems as disclosed in the above-mentioned prior arts. The present invention was developed in consideration of these needs.

### SUMMARY OF THE INVENTION

In a first aspect, the invention provides a distributed sterilizer system for distributing ozone water to a plurality of rooms comprising:
an ozone generation system for generating ozone from an air supply;
a plurality of injection devices for mixing the ozone generated from the ozone generation system with a supply of water to form ozone water; and
a delivery piping system for distributing the ozone water from the plurality of injection devices into each of the plurality of rooms;
wherein the ozone generation system further comprises:
   a centralized oxygen concentrator for providing a supply of oxygen; and
   a plurality of ozone generators for generating ozone from the oxygen supplied by the centralized oxygen concentrator.

The present invention seeks to provide a distributed sterilizer system for distributing ozone water to a plurality of rooms in an efferent manner. At each stages of the sterilizer system, from generation of ozone to the distribution of ozone water, a system controller is adapted to optimize the efficiency of the sterilizer system.

The distributed sterilizer system provides an ozone generation system for generating ozone from an air supply which comprises oxygen. In the ozone generation system, the air supply is directed to the centralized oxygen concentrator which then supplies a supply of concentrated oxygen to the plurality of ozone generators through a plurality of oxygen flow meters. The ozone generated from the plurality of ozone generators is distributed separately and simultaneously to each of the plurality of injection devices for forming ozone water. In the plurality of injection devices, the supply of generated ozone is mixed with a supply of water to form ozone water. The ozone water formed in the plurality of injection devices is then distributed to the plurality of rooms through a dedicated delivery piping system. An incoming water supply system is equipped to supply water to the plurality of injection devices to facilitate the mixing of water and ozone to form ozone water. A plurality of water outlets is connected to the plurality of injection devices which act as a trigger mechanism to initiate the forming of ozone water in the injection devices.

In another embodiment, the distributer sterilizer system is also configured to supply and distribute ozone directly to a plurality of rooms. The ozone generated from the ozone generation system is distributed to the plurality of rooms through a series output tubing and a plurality of air nozzles.

In another embodiment, a system controller is adapted for controlling and monitoring the production of ozone in the ozone generation system, the production of ozone water in the injection devices and the distribution of the supply of ozone water and ozone. The system controller is connected to the ozone generation system and the plurality of injection devices which allows the system controller to control the generation and distribution of ozone and ozone water in the distributed sterilizer system.

In another embodiment, a plurality of flow switches is equipped in the plurality of injection devices for detecting a flow of water in the injection devices. When a user opens a water outlet in any of the specific rooms, water starts to flow from the incoming water supply to the specific injection device. Upon detecting the flow of water in the injection device, the flow switch sends signals to the system controller to send signals to the ozone generation system to generate ozone. The specific ozone generator in the ozone generation system generates ozone using the supply of oxygen from the centralized oxygen concentrator. The ozone generated from the ozone generator is supplied to the specific injection device to initiate the mixing of the ozone with the flow of water in the injection device to form ozone water. The ozone water formed in the injection device is distributed to the specific room in which the water outlet is opened.

In another embodiment, a timer is incorporated for selectively operating components of the ozone generation system between on and off states. The timer is configured to define the duration of the on and off states in which the components in the ozone generation system are switched off for a predetermined time interval before the components restart again. The components of the ozone generation system remain switched on for a predetermined time interval before the components are switched off.

In another embodiment, the forming and distribution of ozone water to a plurality of rooms is determined by a demand-based configuration. The demand-based configuration is configured to be dependent on the demand for ozone water by the user in any of the plurality of rooms. Each of the injections devices is equipped with a flow switch. When a user opens a water outlet in any of the specific rooms, water from the incoming water supply starts to flow into the specific injection device. Upon detecting the flow of water in the injection device, the flow switch sends signals to the system controller to send signals to the specific ozone generator in the ozone generation system to generate ozone. The ozone generated from the ozone generator is supplied to the specific injection device to initiate the mixing of the ozone with the flow of water in the injection device to form ozone water. The ozone water formed in the injection device is distributed to the specific room in which the water outlet is opened.

In another embodiment, the forming and distribution of ozone water to a plurality of rooms is determined by a similar demand-based configuration. This embodiment accommodates the feature of generating and supplying ozone generated from the ozone generation system directly to a plurality of other rooms according to a time-based configuration. In this embodiment, an ozone generator in the ozone generation system serves as a dedicated ozone generator for supplying ozone directly to the plurality of rooms.

Each of the injection devices is equipped with a flow switch. When a user opens a water outlet in any of the specific rooms, water from the incoming water supply starts to flow into the specific injection device. Upon detecting the flow of water in the injection device, the flow switch sends signals to the system controller to send signals to the specific ozone generator to generate ozone. The ozone generated from the ozone generator is supplied to the specific injection device to initiate the mixing of the ozone with the flow of water in the injection device to form ozone water. The ozone water formed in the injection device is distributed to the specific room in which a flow of water is detected in the injection device.

The generation of ozone to be supplied directly to the plurality of rooms is determined by a time-based configuration which is controlled by the timer incorporated in the system controller. According to a predetermined time interval as configured in the timer, the system controller sends signals to the centralized oxygen concentrator in the ozone generation system to supply oxygen to the dedicated ozone generator to generate ozone. The generated ozone in the dedicated ozone generator is supplied directly to the plurality of rooms.

In another embodiment, the forming and distribution of ozone water to a plurality of rooms is determined by a similar demand-based configuration. This embodiment accommodates the feature of generating and supplying ozone generated from the ozone generation system directly to a plurality of other rooms according to a time-based configuration. In this embodiment, an ozone generator in the ozone generation system serves as a shared ozone generator for supplying ozone directly to the plurality of rooms and to an injection device for forming ozone water.

When a user opens a water outlet in any of the specific rooms, water from the incoming water supply starts to flow to the specific injection device equipped with a flow switch. Upon detecting the flow of water in the injection device, the flow switch sends signals to the system controller to send signals to the specific ozone generator to generate ozone. The ozone generated from the ozone generator is supplied to the specific injection device to initiate the mixing of the ozone with the flow of water in the injection device to form ozone water. The ozone water is distributed to the specific room in which a flow of water is detected in the injection device.

According to a predetermined time interval as configured in the timer, the system controller sends signals to the centralized oxygen concentrator in the ozone generation system to supply oxygen to the shared ozone generator to generate ozone. The ozone generated from the shared ozone generator is supplied directly to the plurality of rooms and the injection device. When a user opens the water outlet in any of the specific rooms, water from the incoming water supply starts to flow into the specific injection device. The available supply of the ozone generated from the shared ozone generator is mixed with the flow of water in the injection device to form ozone water. In the event that the water outlet is closed and no water flows into the injection device, the unused ozone generated from the shared ozone generator is channeled to the plurality of rooms through an output tubing.

In another embodiment, the forming and distribution of ozone water to a plurality of rooms and ozone to a plurality of other rooms is determined by a time-based configuration. In this embodiment, an ozone generator in the ozone generation system serves as a dedicated ozone generator for supplying ozone directly to the plurality of rooms.

According to a predetermined time interval as configured by a timer incorporated in the system controller, the system controller sends signals to the centralized oxygen concentrator to supply oxygen to the plurality of ozone generators and one dedicated ozone generator to generate ozone. The supply of ozone generated from a plurality of ozone generators and one dedicated ozone generator is distributed to the plurality of injection devices and directly to the plurality of rooms, respectively. When a user opens the water outlet, water from the incoming water supply starts to flow into the specific injection device. The available supply of the ozone generated from the ozone generator is mixed with the flow of water in the injection device to form ozone water. In the event that the water outlet is closed and no water flows into the injection device, the unused ozone generated from the ozone generators is channeled to the plurality of rooms through an output tubing.

In another embodiment, the forming and distribution of ozone water to a plurality of rooms and ozone to a plurality of other rooms is determined by a time-based configuration. In this embodiment, an ozone generator in the ozone generation system serves as a shared ozone generator for supplying ozone directly to the plurality of rooms and to an injection device for forming ozone water.

According to a predetermined time interval as configured by a timer incorporated in the system controller, the system controller sends signals to the centralized oxygen concentrator to supply oxygen to the plurality of ozone generators and one shared ozone generator to generate ozone. This supply of ozone generated from a plurality of ozone generators and one shared ozone generator is available to be distributed to the plurality of injection devices and directly to the plurality of rooms, respectively. The supply of ozone generated from the ozone generator is shared and distributed to the plurality of rooms and an injection device. When a user opens the water outlet, water from the incoming water supply starts to flow into the specific injection device. The available supply of the ozone generated from the ozone generator is mixed with the flow of water in the injection device to form ozone water. In the event that the water outlet is closed and no water flows into the injection device, the unused ozone generated from the ozone generators is channeled to the plurality of rooms through an output tubing.

### BRIEF DESCRIPTION OF THE DRAWINGS

The present invention will become more clearly understood from the following description of the embodiments thereof, when taken in conjunction with the accompanying drawings. However, the embodiments and the drawings are given only for the purpose of illustration and explanation, and are not to be taken as limiting the scope of the present invention, the scope of which is to be determined by the appended claims.

In the accompanying drawings, like reference numerals are used to denote like parts throughout the several views.

FIG. 1 is a block diagram of a sterilizer system for distributing ozone water according to a first embodiment;

FIG. 2 is a block diagram of a sterilizer system for distributing ozone water and ozone according to a second embodiment;

FIG. 3 is a block diagram of a sterilizer system for distributing ozone water and ozone according to the third embodiment;

FIG. 4 is a block diagram of a sterilizer system for distributing ozone water and ozone according to the fourth embodiment;

FIG. 5 is a block diagram of a sterilizer system for distributing ozone water and ozone according to the fifth embodiment; and

FIG. 6 are close-up views of an injection device which functions based on a demand-based configuration in FIG. 6A and an injection device which functions based on a time-based configuration in FIG. 6B.

### DETAILED DESCRIPTION OF THE EMBODIMENTS

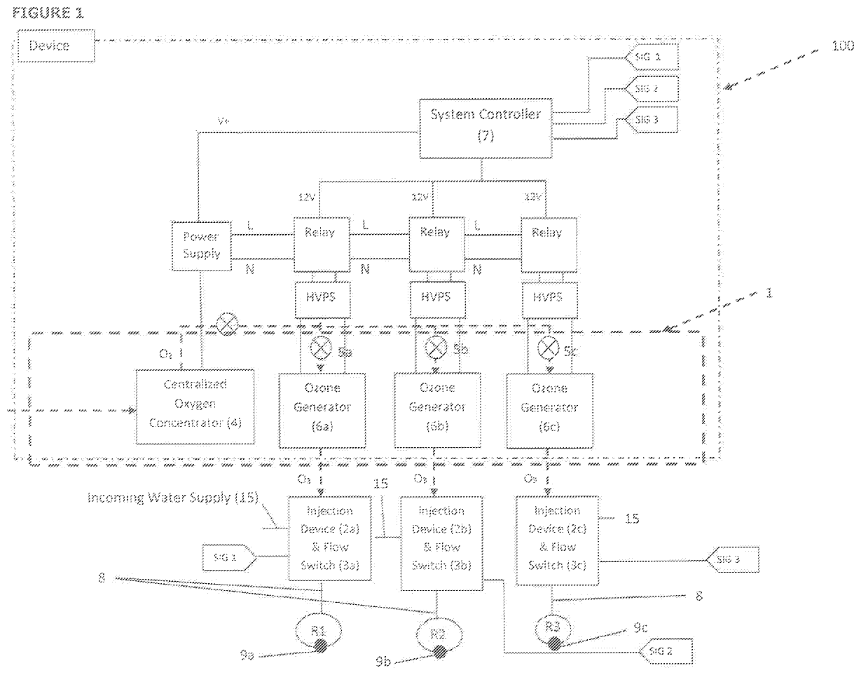

FIG. 1 is a block diagram which illustrates a first embodiment of a distributed sterilizer system 100 for distributing ozone water to a plurality of rooms R1, R2, R3 which has an ozone generation system 1, a plurality of injection devices 2a, 2b, 2c, a plurality of flow switches 3a, 3b, 3c, a plurality of water outlets 9a, 9b, 9c and a delivery piping system 8. The plurality of rooms, R1, R2, R3 are different sections in a kitchen area in which ozone water is used for washing in the washing section, butchery section and food preparation section.

The ozone generation system 1 comprises a centralized oxygen concentrator 4, three oxygen flow meters 5a, 5b, 5c, and three ozone generators 6a, 6b, 6c. The outdoor air is directed to the ozone generation system 1 in which it is first directed to the centralized oxygen concentrator 4. In the centralized oxygen concentrator 4, the supply of outdoor air containing 21% of oxygen combined with nitrogen and a mixture of other gases. The air supply is then pressurized and compressed in the centralized oxygen concentrator 4 to yield oxygen with a pressure in the range of 0.04 MPa to 0.06 MPa. The examples of the different ranges of the oxygen purity/concentration generated from the centralized oxygen concentrator 4 are as follows: 55%@3 LPM, 70%@2 LPM and 90%@1 LPM.

In this embodiment, the forming and distribution of ozone water to a plurality of rooms R1, R2, R3 is determined by a demand-based configuration. Each of the injection devices 2a, 2b, 2c is equipped with a flow switch 3a, 3b, 2c, respectively. When a user opens any of the water outlets 9a, 9b, 9c in any of the specific rooms R1, R2, R3, water starts to flow from an incoming water supply 15 to the injection devices 2a, 2b, 2c. Upon detecting the flow of water in the injection devices 2a, 2b, 2c, the flow switches 3a, 3b, 3c send signals to the system controller 7 to send signals to the ozone generation system 1. Upon receiving signals from the system controller 7, the centralized oxygen concentrator 4 in the ozone generation system 1 start supplying oxygen to the ozone generators 6a, 6b, 6c through the plurality of oxygen flow meters, 5a, 5b, 5c respectively, to generate ozone. The ozone generated from the ozone generators 6a, 6b, 6c is supplied to the injection devices 2a, 2b, 2c to initiate the mixing of the ozone with the flow of water in the injection devices 2a, 2b, 2c to form ozone water. The ozone water formed in the injection devices 2a, 2b, 2c is distributed to the rooms R1, R2, R3 in which a flow of water is detected in the respective injection devices 2a, 2b, 2c. The ozone water is distributed to the plurality of rooms R1, R2, R3 through the delivery piping system 8.

For example, when a user opens a water outlet 9a in room R1, water from the incoming water supply 15 starts to flow to the injection device 2a. Upon detecting the flow of water in the injection device 2a, the flow switch 3a sends signals to the system controller 7 to send signals to the centralized oxygen concentrator 4 to supply oxygen to the ozone generator 6a to generate ozone. The ozone generated from the ozone generator 6a is supplied to the injection device 2a to initiate the mixing of the ozone with the flow of water in the injection device 2a to form ozone water. The ozone water formed in the injection device 2a is distributed to room R1 through the delivery piping system 8.

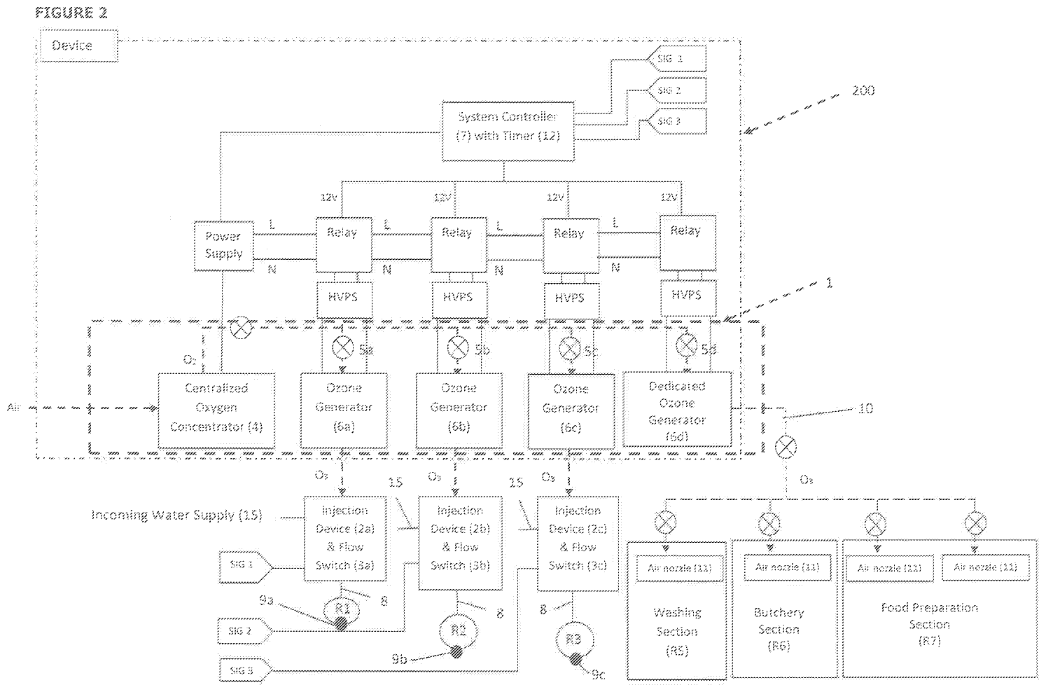

FIG. 2 is a block diagram which illustrates a second embodiment of a distributed sterilizer system 200 which comprises all the components of the first embodiment with the addition of a dedicated ozone generator 6d in the ozone generation system 1, an oxygen flew meter 5d and a timer 12 as incorporated in the system controller 7. In this embodiment, the forming and distribution of ozone water to a plurality of rooms R1, R2, R3 is determined by a similar demand-based configuration as illustrated in FIG. 1. This embodiment accommodates the feature of generating and supplying ozone generated from the ozone generation system 1 directly to a plurality of other rooms R5, R6, R7 according to a time-based configuration using a timer 12 as incorporated in the system controller 7. In this embodiment, an ozone generator 6d in the ozone generation system 1 serves as a dedicated ozone generator for supplying ozone directly to the plurality of other rooms R5, R6, R7. The plurality of other rooms, R5, R6, R7 are different sections in a kitchen area (washing section, butchery section and food preparation section) in which ozone is used to sterilize the surrounding air in the rooms

The timer 12 selectively operates the dedicated ozone generator 6d of the ozone generation system 1 between on and off states by defining the duration of the time intervals for the OFF and ON states. According to the time interval, the dedicated ozone generator 6d generates and supplies ozone directly to the plurality of other rooms R5, R6, R7. The duration of the time intervals is configured by the user. For example, in this embodiment, the interval time is 15 minutes. The dedicated ozone generator 6d is switched OFF for an interval time of 15 minutes. After the interval 15 minutes ends, the dedicated ozone generator 6d is switched ON again for the next 15 minutes before it is switched OFF again.

During the interval time of 15 minutes in which the dedicated ozone generator 6d is switched ON, the system controller 7 sends signals to the centralized oxygen concentrator 4 in the ozone generation system 1 to supply oxygen to the dedicated ozone generator 6d through an oxygen flow meter 5d. The dedicated ozone generator 6d generates a supply of ozone to be distributed directly to the plurality of other rooms R5, R6, R7 through a series of output tubing 10 and air nozzles 11.

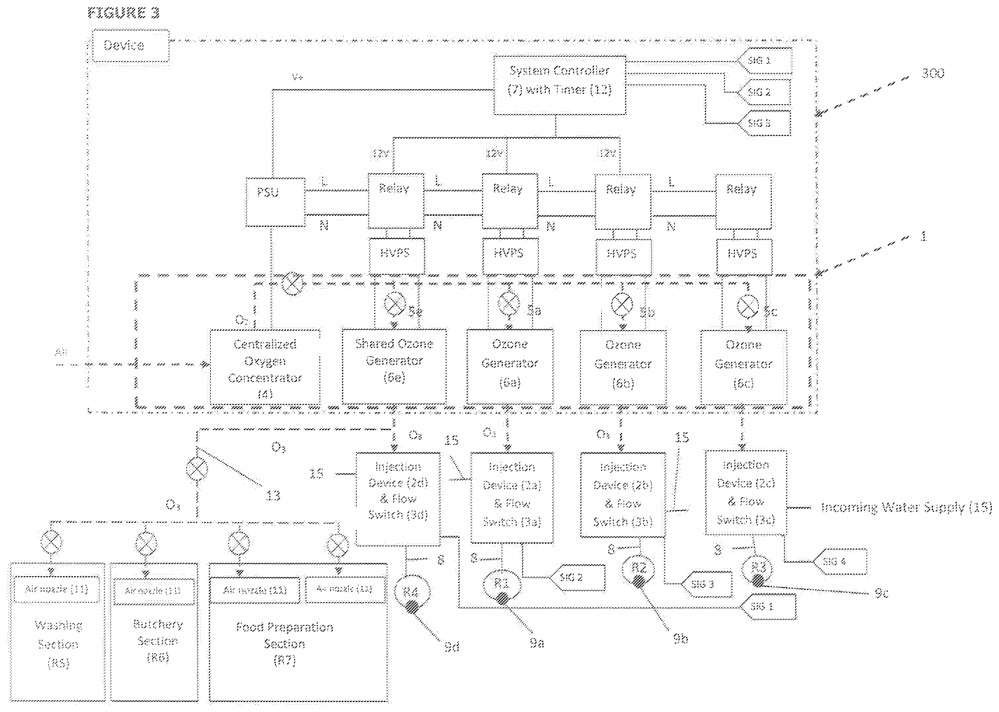

FIG. 3 is a block diagram which illustrates a third embodiment of a distributed sterilizer system 300 which comprises all the components of the first embodiment and an addition of a shared ozone generator 6e in the ozone generation system 1, an oxygen flow meter 5e, an injection device 2d, a flow switch 3d and a timer 12. In this embodiment, the forming and distribution of ozone water to a plurality of rooms R1, R2, R3, R4 is determined by a similar demand-based configuration as illustrated in FIG. 1. This embodiment accommodates the feature of generating and supplying ozone generated from the ozone generation system 1 directly to a plurality of other rooms R5, R6, R7 according to a time-based configuration using a timer 12 as incorporated in the system controller 7. In this embodiment, an ozone generator 6e in the ozone generation system 1 serves as a shared ozone generator for supplying ozone directly to the plurality of other rooms R5, R6, R7 and to an injection device 2d.

The timer 12 selectively operates the shared ozone generator 6e of the ozone generation system 1 between on and off states by defining the duration of the time intervals for the OFF and ON states. According to the time interval, the shared ozone generator 6e generates and supplies ozone directly to the plurality of other rooms R5, R6, R7 and to an injection device 2d. The duration of the time intervals is configured by the user. For example, in this embodiment, the interval time is 15 minutes. The shared ozone generator 6e is switched OFF for an interval time of 15 minutes. After the interval 15 minutes ends, the shared ozone generator 6e is switched ON again for the next 15 minutes before it is switched OFF again.

During the interval time of 15 minutes in which the shared ozone generator 6e is switched ON, the system controller 7 sends signals to the centralized oxygen concentrator 4 to supply oxygen to the shared ozone generator 6e through the oxygen flow meter 5e. The shared ozone generator 6e generates a supply of ozone to be distributed directly to the plurality of other rooms R5, R6, R7 and to the injection device 2d for forming ozone water to be supplied to the room R4. The supply ozone generated from the shared ozone generator 6e is supplied directly to the plurality of other rooms R5, R6, R7 through a series of output tubing 13 and air nozzles 11. The ozone distributed from the shared ozone generator 6e to the injection device 2d remains available in the injection device 2d for the process of mixing subjected to a demand by the user in room R4. When a user opens the water outlet 9d in the room R4, water starts to flow from the incoming water supply 15 into the injection device 2d. The available supply of the ozone generated from the shared ozone generator 6e is mixed with the flow of water in the injection device 2d to form ozone water. In the event that the water outlet 9d is closed in the room R4, and no water flows into the injection device 2d, the unused ozone remaining in the injection device 2d generated from the shared ozone generator 6e is channeled to the plurality of other rooms R5, R6, R7 through an output tubing 13.

In this third embodiment, the forming and distribution of ozone water to a plurality of rooms R1, R2, R3, R4 is determined by a similar demand-based configuration as illustrated in FIG. 1. When a user opens any of the water outlet 9a, 9b, 9c, 9d in any of the specific rooms R1, R2, R3, R4 water starts to flow from the incoming water supply 15 to its respective injection devices 2a, 2b, 2c, 2d. Upon detecting the flow of water in the injection devices 2a, 2b, 2c, 2d, the flow switches 3a, 3b, 3c, 3d send signals to the system controller 7 to send signals to the ozone generator 6a, 6b, 6c and the shared ozone generator 6e to generate ozone. The ozone generated from the ozone generators 6a, 6b, 6c and the shared ozone generator 6e is supplied to the injection devices 2a, 2b, 2c, 2d respectively to initiate the mixing of the ozone with the flow of water in the injection devices 2a, 2b, 2c, 2d to form ozone water. The ozone water formed in the injection devices 2a, 2b, 2c, 2d is distributed to the rooms R1, R2, R3, R4 respectively in which a flow of water is detected in the respective injection devices 2a, 2b, 2c, 2d. The ozone water is distributed to the plurality of rooms R1, R2, R3, R4 through the delivery piping system 8.

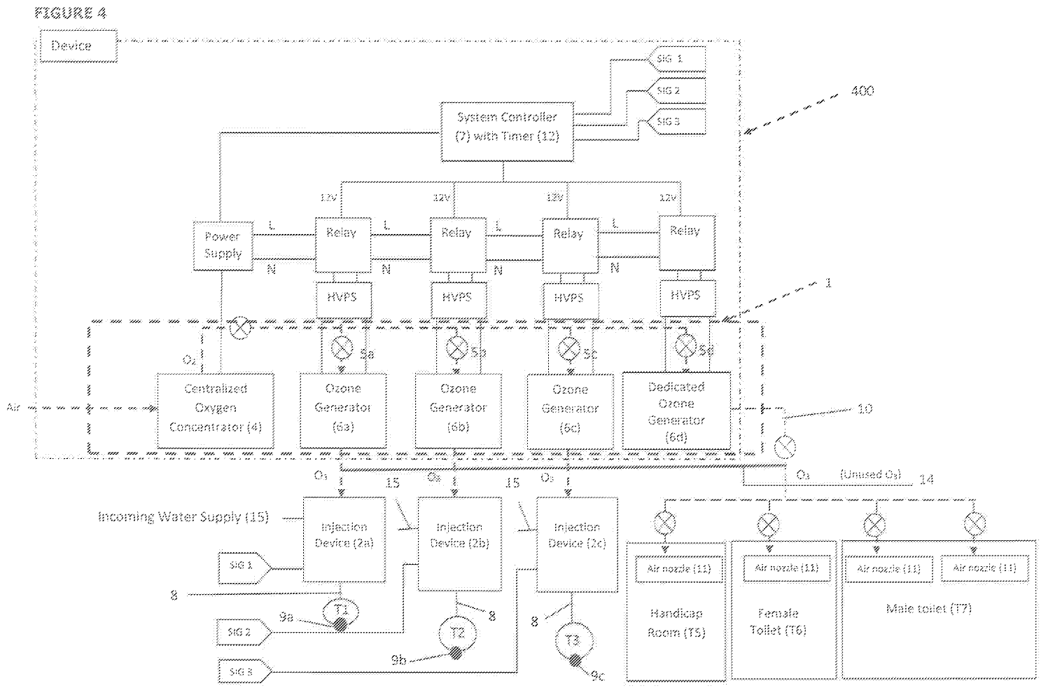

FIG. 4 is a block diagram which illustrates a fourth embodiment of a distributed sterilizer system 400 which comprises all the components of the second embodiment for providing ozone water to a plurality of rooms T1, T2, T3 except the plurality of flow switches and an addition of an output tubing 14 for distributing unused ozone from the ozone generators, 6a, 6b, 6c to the plurality of other rooms T5, T6, T7. In this embodiment, the forming and distribution of ozone water to a plurality of rooms T1, T2, T3 and ozone to a plurality of other rooms T5, T6, T7 is determined by a time-based configuration using the timer 12. The plurality of rooms T1, T2, T3, are washrooms in which the ozone water is used for cleaning in the toilet bowls, basins, bidets and urinals. The plurality of other rooms T5, T6, T7 are washrooms in which the ozone is used to sterilize the surrounding air in the rooms. In this embodiment, an ozone generator 6d in the ozone generation system 1 serves as a dedicated ozone generator for supplying ozone directly to the plurality of other rooms T5, T6, T7 through the series of output tubing 10 and air nozzles 11.

The timer 12 selectively operates the plurality of ozone generators 6a, 6b, 6c and the dedicated ozone generator 6d of the ozone generation system 1 between on and off states by defining the duration of the time intervals for the OFF and ON states. According to the time interval, the plurality of ozone generators 6a, 6b, 6c and the dedicated ozone generator 6d generates and supplies ozone to the plurality of injection devices 2a, 2b, 2c and directly to the plurality of rooms T5, T6, T7, respectively.

The duration of the time intervals is configured by the user. For example, in this embodiment, the interval time is 15 minutes. The ozone generators 6a, 6b, 6c and the dedicated ozone generator 6d are switched OFF for an interval time of 15 minutes. After the interval 15 minutes ends, the ozone generators 6a, 6b, 6c and the dedicated ozone generator 6d are switched ON again for the next 15 minutes before it is switched OFF again.

During the interval time of 15 minutes in which the ozone generators 6a, 6b, 6c and the dedicated ozone generator 6d are switched ON, the system controller 7 sends signals to the centralized oxygen concentrator 5 to supply oxygen to the ozone generators 6a, 6b, 6c and the dedicated ozone generator 6d. The ozone generators 6a, 6b, 6c and the dedicated ozone generator 6d generate a supply of ozone to be supplied and distributed to the plurality of injection devices 2a, 2b, 2c and directly to the plurality of rooms T5, T6, T7, respectively. The ozone distributed from the ozone generator 6a, 6b, 6c to the injection device 2a, 2b, 2c remains available in the injection devices 2a, 2b, 2c respectively for the process of mixing subjected to a demand by the users in any of the respective rooms T1, T2, T3. At any time during the interval time of 15 minutes, when a user opens any of the water outlets 9a, 9b, 9c in the plurality of rooms, T1, T2, T3 respectively, water from the incoming water supply 15 starts to flow into the injection devices 2a, 2b, 2c. The available supply of the ozone generated from the ozone generators 6a, 6b, 6c at the interval time of 15 minutes is mixed with the flow of water in the injection devices 2a, 2b, 2c to form ozone water. The ozone water from the injection devices 6a, 6b, 6c is distributed to the water outlets 9a, 9b, 9c accordingly. In the event that the water outlets 9a, 9b, 9c are closed at any time during the interval time of 15 minutes and no water flows into the injection devices 2a, 2b, 2c, the unused ozone generated from the ozone generators 6a, 6b, 6c is channeled to the plurality of other rooms T5, T6, T7 through an output tubing 14.

For example, at any time during the interval time of 15 minutes, when a user opens a water outlet 9a in room T1, water from the incoming water supply 15 starts to flow into the injection device 2a. The supply of the ozone generated from the ozone generator 6a at the interval time of 15 minutes is mixed with the flow of water in the injection device 2a to form ozone water. The ozone water is supplied to the water outlet 9a. In the event that the water outlet 9a is closed at any time during the interval time of 15 minutes and no water flows into the injection device 2a, the unused ozone generated from the ozone generator 6a is channeled to the plurality of other rooms T5, T6, T7 through an output tubing 14.

During the interval time of 15 minutes in which the dedicated ozone generator 6d is switched ON, the dedicated ozone generator 6d generates a supply of ozone to be distributed directly to the plurality of other rooms T5, T6, T7 through the series of tubing 10 and air nozzles 11.

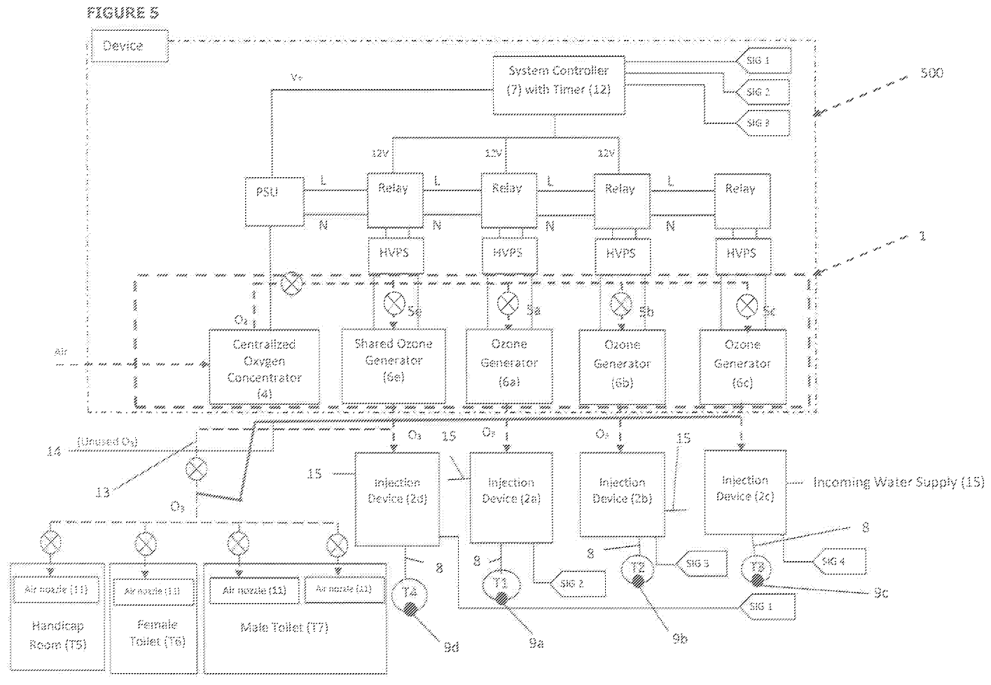

FIG. 5 is a block diagram which illustrates a fifth embodiment of a distributed sterilizer system 500 which comprises all the components of the third embodiment for providing ozone water to a plurality of rooms T1, T2, T3 except the plurality of flow switches and an addition of an output tubing 14 for distributing unused ozone to the plurality of other rooms T5, T6, T7. In this embodiment, the forming and distribution of ozone water to a plurality of rooms T1, T2, T3 and ozone to a plurality of other rooms T5, T6, T7 is determined by a time-based configuration using the timer 12. The plurality of rooms T1, T2, T3, are washrooms in which the ozone water is used for cleaning in the toilet bowls, basins, bidets and urinals. The plurality of other rooms T5, T6, T7 are washrooms in which the ozone is used to sterilize the surrounding air in the rooms. In this embodiment, an ozone generator 6e in the ozone generation system 1 serves as a shared ozone generator tor supplying ozone directly to the plurality of other rooms T5, T6, T7 and to an injection device 2d.

During the interval time of 15 minutes in which the ozone generators 6a, 6b, 6c and the shared ozone generator 6e are switched ON, the system controller 7 sends signals to the centralized oxygen concentrator 5 to supply oxygen to the ozone generators 6a, 6b, 6c and the shared ozone generator 6e through plurality of oxygen flow meters, 5a, 5b, 5d, 5e respectively. The ozone generators 6a, 6b, 6c and the shared ozone generator 6e generate a supply of ozone to be supplied and distributed to the plurality of injection devices 2a, 2b, 2c, 2d and directly to the plurality of rooms T5, T6, T7, respectively. The supply of ozone generated from the shared ozone generator 6e is shared and distributed to the plurality of other rooms T5, T6, T7 through the series of output tubing 13 and air nozzles 11 and to the injection device 2d for supplying ozone water to the room T4.

At any time during the interval time of 15 minutes, when a user opens any of the water outlets 9a, 9b, 9c, 9d in the plurality of rooms, T1, T2, T3, T4 respectively, water starts to flow from the incoming water supply 15 into the injection devices 2a, 2b, 2c, 2d. The supply of the ozone generated from the ozone generators 6a, 6b, 6c and the shared ozone generator 6e at the interval time of 15 minutes is mixed with the flow of water in the injection devices 2a, 2b, 2c, 2d respectively to form ozone water. The ozone water is distributed to the water outlets 9a, 9b, 9c, 9d accordingly. In the event that any of the water outlets 9a, 9b, 9c, 9d are closed at any time during the interval time of 15 minutes and no water flows into the injection devices 2a, 2b, 2c, 2d, the unused ozone generated from the ozone generators 6a, 6b, 6c and the shared ozone generator 6e is channeled to the plurality of other rooms T5, T6, T7 through an output tubing 14.

For example, at any time during the interval time of 15 minutes, when a user opens a water outlet 9d in room T4, water from the incoming water supply 15 starts to flow into the injection device 2d. The available supply of the ozone generated from the shared ozone generator 6e at the interval time of 15 minutes is mixed with the flow of water in the injection device 2d to form ozone water. The ozone water is supplied to the water outlet 9d. In the event that the water outlet 9d is closed at any time during the interval time of 15 minutes and no water flows into the injection device 2d, the unused ozone generated from the ozone generator 6e is channeled to the plurality of other rooms T5, T6, T7 through on output tubing 14.

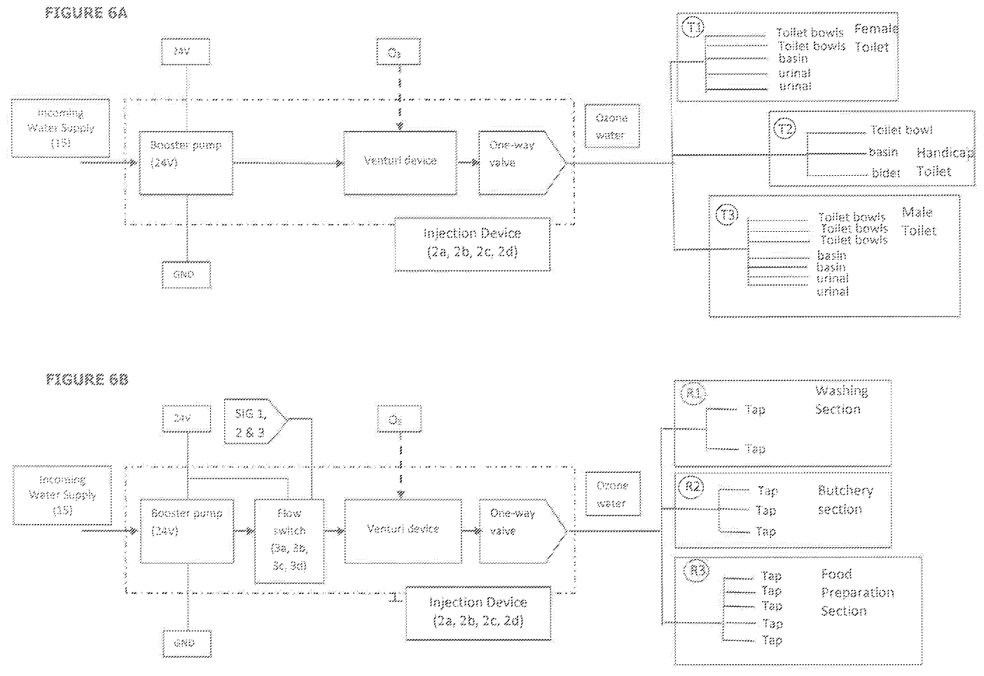

FIG. 6 illustrates close-up views of injection devices in which FIG. 6A illustrates an injection device which functions based on a time-based configuration in FIG. 6A and FIG. 6B illustrates an injection device which functions based on a demand-based configuration.

FIG. 6A illustrates a close-up view of an injection device 2a, 2b, 2c, 2d which is used in the fourth and fifth embodiments which are based on a time-based configuration. The injection device in FIG. 6A facilitates the mixing of a supply of water from the incoming water supply 15 with the ozone generated from the ozone generator (shown in FIG. 4 or 5) according to a predetermined time-interval to form ozone water. The ozone water is distributed to the plurality of rooms T1, T2, T3. The plurality of rooms, T1, T2, T3 are washrooms in which the ozone water is used for cleaning in the toilet bowls, basins, bidets and urinals. When a water outlet (shown in FIG. 4 or 5) in any of the rooms T1, T2, T3 is opened, a supply of water from the incoming water supply 15 flows into a venturi device in the injection device 2a, 2b, 2c, 2d. This supply of water in the venturi device is available for mixing with the ozone generated from the ozone generator (shown in FIG. 4 or 5). The supply of ozone is generated based on a pre-determined time interval in which the system controller (shown in FIG. 4 or 5) sends signals to the ozone generation system (shown in FIG. 4 or 5) to generate ozone. The ozone is distributed into the venturi device in the injection device 2a, 2b, 2c, 2d for the mixing process to form ozone water. In the event, no water outlet is opened in any of the rooms T1, T2, T3, the unused ozone will remain in the venturi device and eventually be channeled to any of the other plurality of rooms (shown in FIG. 4 or 5). In between the time-interval in which the ozone generation system (shown in FIG. 4 or 5) is switched OFF, the opening of a water outlet in any of the rooms T1, T2, T3 during this time triggers the flow of water into the venturi device to mix with the remaining unused ozone supplied from the previous cycle to form ozone water.

FIG. 6B illustrates a close-up view of an injection device 2a, 2b, 2c, 2d which is used in the first, second and third embodiments which are based on a demand-based configuration. The injection device 2a, 2b, 2c, 2d is incorporated with a flow switch 3a, 3b, 3c, 3d which facilitates the mixing of a supply of water from the incoming water supply 15) with the ozone generated from the ozone generator (shown in FIG. 1, 2 or 3) to form ozone water. The ozone water is distributed to the plurality of rooms R1, R2, R3. The plurality of rooms, R1, R2, R3 are sections in a kitchen area in which the ozone water is used for washing in the washing section, butchery section and food preparation section. When a water outlet (shown in FIG. 1, 2 or 3) in any of the rooms R1, R2, R3 is opened, a supply of water the incoming water supply 15) flows into a venturi device in the injection device 2a, 2b, 2c, 2d. The flow of water into the venturi device triggers the flow switch 3a, 3b, 3c, 3d to send a signal to the system controller (shown in FIG. 1, 2 or 3) to send signals to the ozone generation system (shown in FIG. 1, 2 or 3) to generate and supply ozone to the injection device 2a, 2b, 2c, 2d. The ozone generated from the ozone generator (shown in FIG. 1, 2 or 3) is distributed to the venturi device for mixing with the supply of water to form ozone water. The ozone water is distributed to the plurality of rooms R1, R2, R3.

The invention may also be embodied in many ways other than those specifically described herein, without departing from the scope thereof.

1. A distributed sterilizer system for distributing ozone water and ozone to a plurality of rooms comprising:
an ozone generation system for generating ozone from an air supply;
a plurality of injection devices for mixing the ozone generated from the ozone generation system with a supply of water to form ozone water;
a delivery piping system for distributing the ozone water from the plurality of injection devices into each of the plurality of rooms; and
a timer for selectively operating components of the ozone generation system between on and off states by defining a duration for at least one of said on and off states;
wherein the ozone generation system further comprises:
a centralized oxygen concentrator for providing a supply of oxygen; and
a plurality of ozone generators for generating ozone from the oxygen supplied by the centralized oxygen concentrator;
wherein the ozone generation system provides a supply of ozone directly into each of a plurality of other rooms.

2. The distributed sterilizer system according to claim 1, wherein the ozone generation system further comprises:
a plurality of oxygen flow meters for distributing the supply of oxygen from the centralized oxygen concentrator into the plurality of ozone generators.

3. The distributed sterilizer system according to claim 1, further comprising an incoming water supply system to supply water to the plurality of injection devices.

4. The distributed sterilizer system according to claim 1, further comprising a plurality of water outlets connected to the injection devices to act as a trigger mechanism for production of ozone water.

5. The distributed sterilizer system according to claim 4, wherein a respective said water outlet is allocated for each room and positioned in the interior of the room.

6. The distributed sterilizer system according to claim 1, further comprising a system controller which controls and monitors the production of ozone in the ozone generation system and the production of ozone water in the plurality of injection devices, and the distribution of the supply of ozone water.

7. (canceled)

8. (canceled)

9. (canceled)

10. The distributed sterilizer system according to claim 1, further comprising a plurality of flow switches for detecting a flow of water in the plurality of injection devices.

11. The distributed sterilizer system according to claim 10, wherein a flow switch is provided for each of the plurality of injection devices.

12. The distributed sterilizer system according to claim 10, further comprising a system controller which controls and monitors the production of ozone in the ozone generation system and the production of oxone water in the plurality of injection devices, and the distribution of the supply of ozone, and wherein upon detecting water in the injection devices, the flow switches send signals to the system controller which subsequently sends signals to the ozone generation system to supply ozone to the injection devices for producing ozone water.

13. The distributed sterilizer system according to claim 7, further comprising a dedicated ozone generator in the ozone generation system to supply and distribute ozone to the plurality of other rooms.

14. The distributed sterilizer system according to claim 1, further comprising a shared ozone generator in the ozone generation system to supply and distribute ozone to the plurality of other rooms and to an injection device for forming ozone water to be supplied to a room.

15. The distributed sterilizer system according to claim 14, wherein when no water is flowing in the injection device because of a closed water outlet, the unused ozone generated from the shared ozone generator is channeled into each of the plurality of other rooms through an output tubing.

16. The distributed sterilizer system according to claim 1, further comprising a system controller that controls and monitors the production of ozone in the ozone generation system, and wherein at predetermined time intervals, the system controller sends signals to the ozone generation system to provide a supply of ozone into the plurality of injection devices.

17. The distributed sterilizer system according to claim 1, further comprising a system controller that controls and monitors the production of ozone in the ozone generation system, and wherein at predetermined time intervals, the system controller sends signals to the ozone generation system to provide a supply of ozone into each of the plurality of other rooms.

18. The distributed sterilizer system according to 16, wherein when no water is flowing in the injection device because of a closed water outlet, the unused ozone generated from the ozone generator is channeled into each of the plurality of other rooms through an output tubing.

19. The distributed sterilizer system according to claim 16, further comprising a dedicated ozone generator in the ozone generation system to supply and distribute ozone to the plurality of other rooms.

20. The distributed sterilizer system according to 16, further comprising a shared ozone generator in the ozone generation system to supply and distribute ozone to the plurality of other rooms and to an injection device for forming ozone water to be supplied to a room.

21. The distributed sterilizer system according to claim 1, further comprising a plurality of air nozzles for distributing ozone generated from the ozone generation system into each of the other rooms.
